(19) United States
(12) Patent Application Publication
(10) Pub. No.: US 2024/0194328 A1
(43) Pub. Date: Jun. 13, 2024

(54) Dual Attention Network Using Transformers for Cross-Modal Retrieval

(71) Applicant: Mayo Foundation for Medical Education and Research, Rochester, MN (US)

(72) Inventors: Hamid R. Tizhoosh, Rochester, MN (US); Danial Maleki, Waterloo (CA)

(21) Appl. No.: 18/536,121

(22) Filed: Dec. 11, 2023

Related U.S. Application Data
(60) Provisional application No. 63/431,644, filed on Dec. 9, 2022.

(51) Int. Cl. G16H 30/40; G06V 10/82; G16H 50/20

(52) U.S. Cl. CPC G16H 30/40; G06V 10/82; G16H 50/20

(57) ABSTRACT

Cross-modal data are extracted from an input data source. A computer system accesses first input data of a first modality and second input data of a second modality. The first and second input data are then input to a dual attention network that has been trained on training data to extract feature data from cross-modal input data. Feature data are generated as outputs by inputting the first and second input data to the dual attention network. The feature data include feature representations of the first modality and the second modality. The feature data can be stored or displayed to a user with the computer system.

### BACKGROUND

The volume of available data has grown dramatically in recent years in many applications. Furthermore, the age of networks that used multiple modalities separately has practically ended. Therefore, enabling bidirectional cross-modality data retrieval capable of processing has become an important goal for many domains and disciplines of research. This is especially true in the medical field, as data comes in a multitude of types, including various types of images and reports, as well as molecular data, genomic data (e.g., RNA, DNA), and the like. Most contemporary works apply cross attention to highlight the essential elements of an image or text in relation to the other modalities and try to match them together. However, regardless of their importance in their own modality, these approaches usually consider features of each modality equally.

### SUMMARY OF THE DISCLOSURE

The present disclosure addresses the aforementioned drawbacks by providing a method for extracting cross-modal data from an input data source. The method includes accessing, with a computer system, first input data of a first modality (e.g., text data corresponding to textual information), and accessing, with the computer system, second input data of a second modality (e.g., image data corresponding to image information). A dual attention network trained on training data to extract feature data from cross-modal input data is also accessed with the computer system. The first input data and the second input data are input to the dual attention network by the computer system, generating outputs as feature data comprising feature representations of the first modality and the second modality. The feature data are stored or displayed to a user with the computer system.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates an example dual attention network that can be implemented for cross-modal retrieval in accordance with some embodiments.

FIG. 2 is an example vision transformer (ViT) network.

FIG. 3 is a diagram of an H-DINO Architecture: illustrating both the student and teacher networks. For the sake of clarity, only one patch per view is represented.

FIG. 4 shows an example of patch extraction and expansion from a whole slide image: The process begins with mask overlay, patch extraction, expansion, and transformation.

FIG. 5 shows an example view selection and augmentation pipeline: Left shows random view selection for Hyper (τ_{H}) and Mild (τ_{M}) augmentation, right displays their respective pipelines with varied intensity levels.

FIG. 6 is an example robustly optimized BERT (Bidirectional Encoder Representations from Transformers) pre-training approach (RoBERTa) network.

FIG. 7 is an example multi-head self-attention module.

FIG. 8 is an example cross attention module.

FIG. 9 illustrates an example of forming a similarity matrix as an output of a dual attention network receiving image data and text data as two inputs. The similarity matrix shown is for a mini-batch with size of n. Element Iᵢ, Tⱼ in the matrix shows the similarity score between image Iᵢ, and text Tⱼ.

FIG. 10 is a flowchart of an example method for implementing a dual attention network to generate feature data from dual modality inputs (e.g., first input data associated with a first modality and second input data associated with a second modality), where the feature data represent cross-modal information.

FIG. 11 shows examples of paired first input data (text) and second input data (images). The images are from different staining and normalizations with varied description structures.

FIG. 12 is a flowchart of an example method for training a dual attention network.

FIG. 13 shows a confusion matrix that depicts the retrieval performance of the proposed method for patch-level classification across 22 distinct primary diagnoses, specifically at an R@5 recall level. It provides a comprehensive visual representation of how accurately the model classifies each diagnosis and demonstrates how the results are distributed across true and predicted classifications, hence offering a deeper understanding of the model's performance.

FIG. 14 shows a confusion matrix that provides a visualization of the retrieval performance at the R@5 level of the proposed method for the WSI-level classification across 22 distinct primary diagnoses. It serves as a detailed display of the model's accuracy in diagnosis classification. By presenting the distribution of results across true and predicted classes, it enables a profound understanding of the overall efficacy and precision of the model.

FIG. 15 shows three randomly selected patches from three distinct Cribriform Carcinoma WSIs present in the GRH dataset. The noticeable differences in structure and the significant variations among these images illuminate the challenge of accurate classification, ultimately contributing to lower accuracy rates.

FIG. 16 illustrates image retrieval using real-world medical descriptions extracted from the WHO dataset, which wasn't included in the original training set. Under each description, the associated primary diagnosis is displayed. To the right, the top three images retrieved by the model are presented alongside their corresponding primary diagnoses. This illustration showcases the model's ability to retrieve and match images based on textual medical descriptions accurately.

FIG. 17 illustrates the Recall@K@V values for various combinations of V and K. In this process, the most relevant primary diagnoses are first sorted for each patch based on a given V value. Subsequently, the Recall@K is computed for each WSI using the frequency of the most common primary diagnosis identified for that WSI. The results of this process are showcased in the figure for multiple voting thresholds (V), which include 1, 2, 3, 5, 10, 15, and 22. The selection of these thresholds is significant as the number of primary diagnoses is capped at 22. Additionally, the figure also displays these results across varying levels of Recall (K), specifically 1, 3, 5, and 10. This visualization provides a comprehensive overview of how alterations in the voting threshold and recall level influence the Recall@K@V values, thereby highlighting the nuanced interplay of these parameters in the evaluation process.

FIG. 18 is a block diagram of an example dual attention network-based cross-modal retrieval system.

FIG. 19 is a block diagram of example components that can implement that system of FIG. 18.

### DETAILED DESCRIPTION

Described here are systems and methods for a dual attention network using transformers for cross-modal information retrieval. As an example, the dual attention network receives first input data associated with a first domain, or modality, and second input data associated with a second domain, or modality, and simultaneously captures features or other information relevant to both domains, or modalities. For instance, the dual attention network can be configured for cross-modal information retrieval in histopathology archives (e.g., the first input data may be histopathology images and the second input data may be text).

Advantageously, the disclosed systems and methods are capable of generating diagnostic reports and retrieving images based on symptom description. Self-attention is used as an additional loss term to enrich the internal representation provided into a cross attention module. In this way, the novel architecture with a new loss term described in the present disclosure can help represent images and texts in a joint latent space.

In some aspects, the present disclosure describes a system of deep networks that can learn and apply images and text at the same time. Advantageously, the disclosed systems and methods are capable of fusing image-text pairs (or other cross-modality data pairs or groups) into a form that can be used to train other machine learning algorithms and improve their output. Additionally or alternatively, other types of data can also be added, such as genomic data (e.g., RNA sequence data, DNA sequence data).

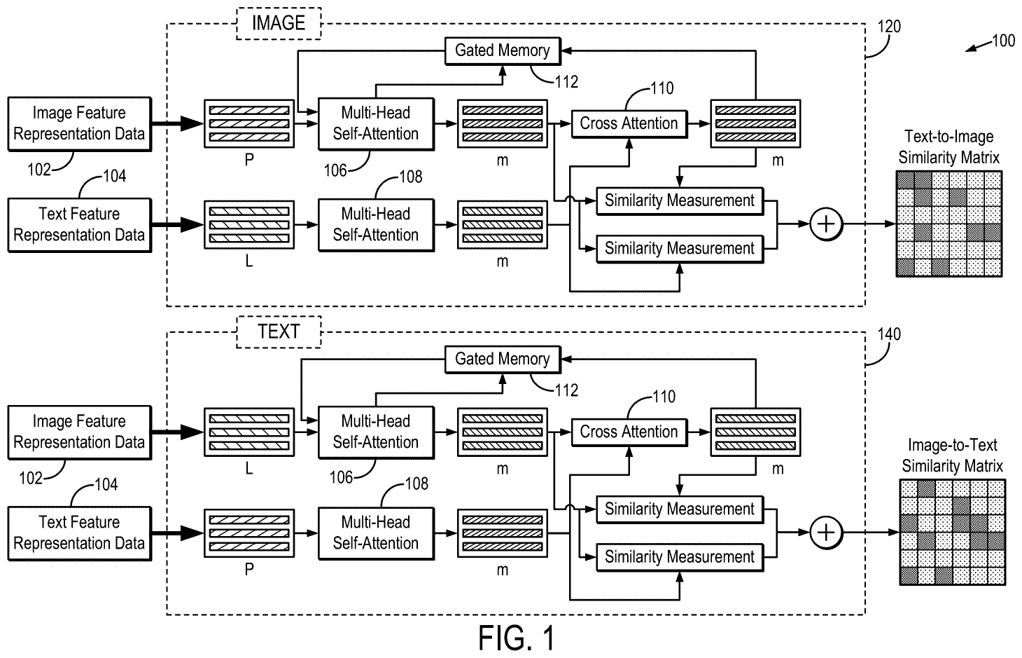

A non-limiting example of a dual attention network that can be used for cross-modal retrieval is shown in FIG. 1. The network may be referred to as a LILE (“Look In-Depth Before Looking Elsewhere”) network. In the illustrated example, a dual attention network 100 includes an image subnetwork 120 and a text subnetwork 140. Although, in other implementations the subnetworks may be replaced with a subnetwork tailored for a different modality, such as genomic data, auditory data, and so on. Each subnetwork 120, 140 receives first feature representation data corresponding to the modality of the subnetwork and second feature representation data corresponding to the modality of the other subnetwork. For instance, as illustrated in FIG. 1, the image subnetwork 120 receives image feature representation data 102 and text feature representation data 104. Similarly, the text subnetwork 140 receives text feature representation data 104 and image feature representation data 102. As a nonlimiting example, the feature representation data (e.g., image feature representation data 102, text feature representation data 104) can be generated using a suitable transformer model.

Then, a self-attention module 106, 108 is applied to extract the most significant parts of each modality with respect to itself. For the next step, a cross-attention module 110 and gated memory block 112 are applied to help each subnetwork 120, 140 refine the representation of each instance with respect to the outputs of the self-attention module for the modality of the subnetwork (i.e., self-attention module 106) and the cross attention module 110. The cross-attention modules 110 are employed to extract important, or otherwise relevant, segments from the first and second input data (e.g., images and text), considering their relevance to the other modality (e.g., text and images, respectively). Additionally, an iterative matching scheme using a gated memory block 112 is applied to refine the extracted features for each modality.

As shown, the image feature representation data 102 and text feature representation data 104 input to the dual attention network 100 may be the outputs of individual transformer models. For example, a transformer architecture can be employed for both image and text encoding backbones, with the self-attention modules of the dual attention network being leveraged to highlight key aspects, or features, of images and text.

Data representation, or representation learning, is a collection of approaches in machine learning that allows a model to automatically discover the representations required from the given data. Recently, transforms have become more prevalent for representation learning. The modular architecture of a transformer model enables the processing of different modalities (e.g., images, videos, text, and voice) leveraging similar processing blocks. Transformer models can scale efficiently to large capacity networks for complex tasks and can perform well with massive datasets, such as WSIs. As noted above, transformer architectures can be used to extract feature representations from the first and second input data (e.g., text and images) in the disclosed systems and methods due to these advantages.

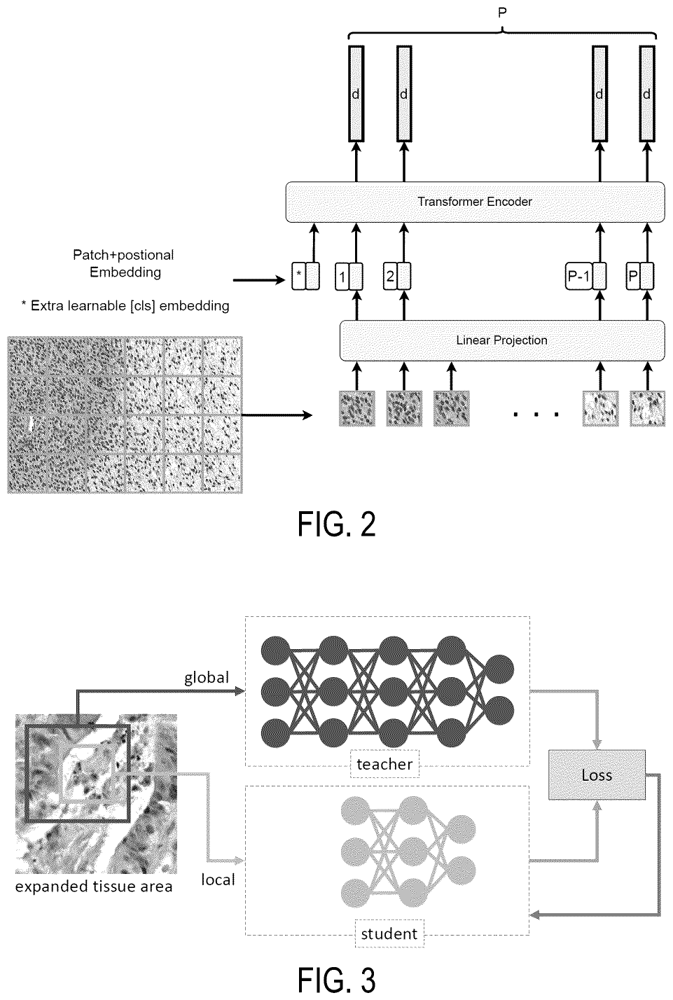

In some implementations, a pre-trained vision transformer (ViT) is implemented to encode input images. An example ViT is illustrated in FIG. 2. In the illustrated example, each image is split into a sequence of fixed-size, non-overlapping image patches before being fed to the ViT. In some other implementations, object detection models may be used to extract visual information (e.g., image feature data) from an image. These models are trained on the input image in order to extract the objects. As a result, the input image can be represented as a set of extracted feature maps for all of the objects in the image. In still other implementations, other techniques for extracting feature data from images may be used to generate image feature data. The choice of feature extraction technique may be chosen depending on the dataset and the availability of annotations for the object detection task.

As another example, a DeiT architecture may be used for an image encoder. This architecture includes 12 layers and 768 hidden state and split the input image into 16×16 patches. As the result, each input image can be seen as 196 patches with the size of 16×16. The DeiT model individually is trained for a classification task on an initial training dataset. In the next step, the weights for the image encoder can be initialized from the trained model in the previous step. When the size of the dataset is not large enough to properly train the network, only the last two blocks of the image encoder may be trained and the rest of the network layers may be frozen.

The scarcity of adequately labeled data for model training is recognized as a prevailing challenge in the medical field. This challenge is notably apparent in the visual modality (e.g., images). To address this, a self-supervised learning (SSL) paradigm to derive image features can be utilized in some embodiments. As a non-limiting example, a distillation with no labels (DINO) SSL approach for a vision transformer can be adapted by introducing a patching technique referred to herein as harmonizing DINO (H-DINO) for whole-slide images (WSIs). This technique is described in more detail below.

In general, a DINO framework creates various crops from a single image. This results in a set, V, holding multiple views of the image, including two global and several local views at reduced resolution. For WSIs and the use of patch extraction in histopathology, this set V can be revised. In a conventional DINO approach, 96×96 local patches and 224×224 global patches may be used. Yet, altering the magnification of patches from a WSI at 20×, which is common in digital pathology, can introduce inconsistencies. To mitigate this, a tailored patching solution named H-DINO, aligning with WSI techniques, can be used. An example of the H-DINO model architecture is shown in FIG. 3.

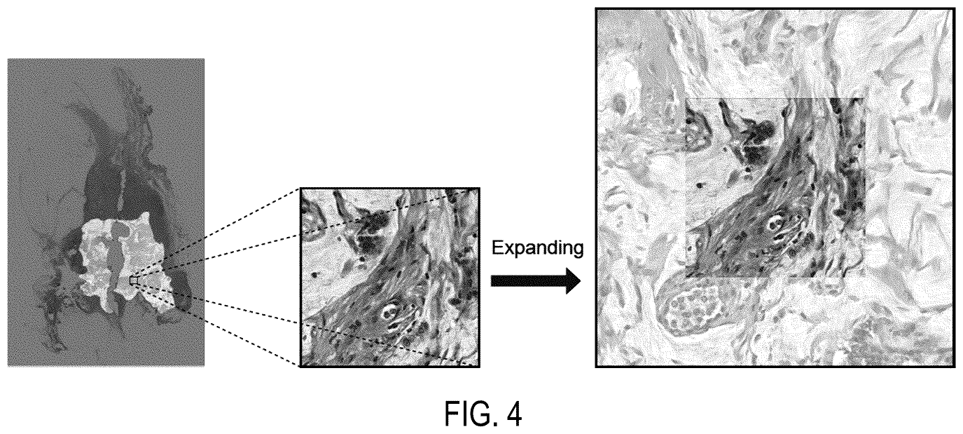

In the H-DINO architecture described here, the extraction of patches is carried out on a scale larger than the final size intended to be used as an input to the network. This is accomplished by enlarging the patch size around the specific location based on the dataset and patching method. The visualization of this step can be seen in FIG. 4. Subsequently, two categories of views (one more global and one more local) are extracted from the expanded patch, with each being subjected to its respective set of transformations. The distinction between these views is determined by the size of the cropped region. As an example, the cropping range for the more local views can be set between 50 and 140 pixels, while for the more global views, the cropping size can be set between 140 and 224 pixels. Given that uniformity in magnification is maintained in all selected patches, the core features of these patches remain unaltered. This consistency ensures that each patch holds its unique representation and characteristics intact, which in turn assists in maintaining the consistency of the information it offers.

Moreover, the uniformity in magnification also guides the learning process for the network in a meaningful way. By processing patches that are expected to share similar attributes, the network learns to map related concepts or visual features closer to each other in its internal representation space. This capacity can advantageously enable the network to distinguish between different classes and categories and, thereby, enable high performance on classification or retrieval tasks.

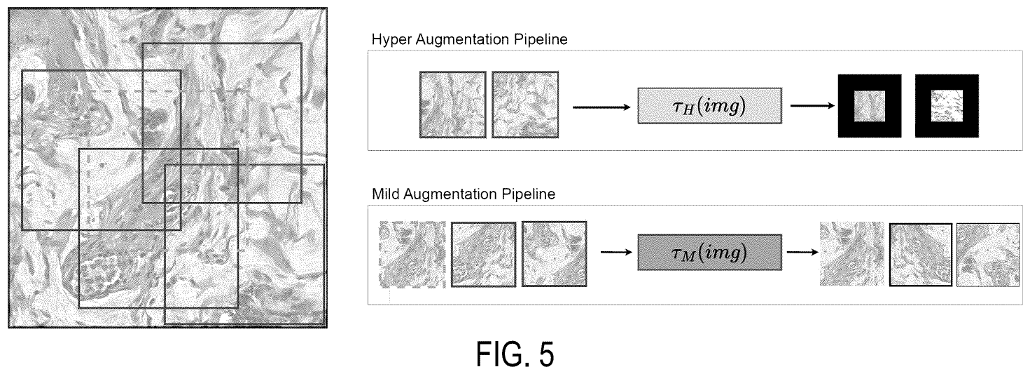

These transformations can be selected to match the specific network into which the patch will be integrated. A comprehensive visualization of this entire process can be found in FIG. 5. This figure depicts the interplay among the extraction, enlargement, and transformation of patches, all contributing to the distinct approach that has been adopted in the design.

After the patch selection, the more robust and less noisy patches are passed through the teacher model, while all the patches that have been generated in the patch selection stage are passed through the student model. Both teacher and student networks are identical in architecture with different sets of parameters: θₜ, and θ_{S} for the teacher and student networks, respectively.

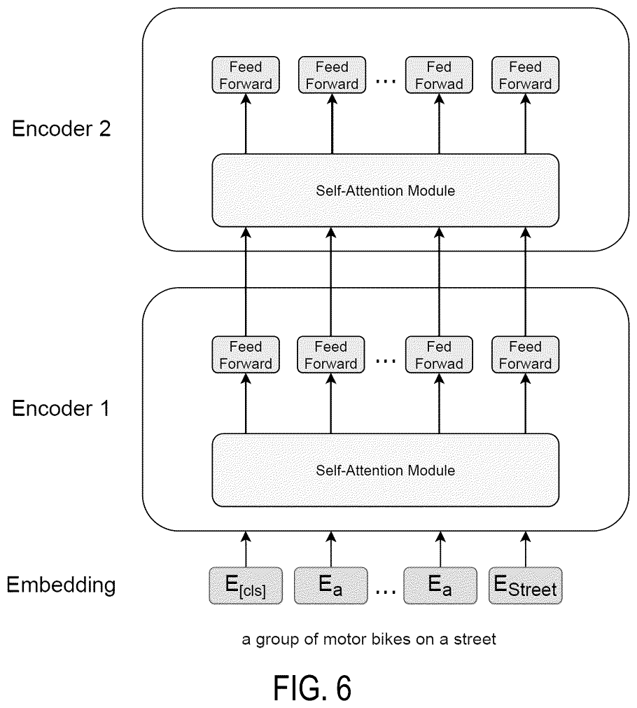

Text representation can generally be defined as a method for encoding sentences into vectors. Transformers can be used for many natural language processing (NLP) tasks. As one example, a bidirectional encoder representations from transformers (BERT) model may be used to generate text feature data. As another example, a robustly optimized BERT approach (RoBERTa) model may be used. A pre-trained RoBERTa architecture includes a transform-based model, which can be deployed to encode the input text. An example RoBERTa architecture is shown in FIG. 6. The RoBERTa architecture receives a text description as an input and returns a feature map that represents the given data.

In a RoBERTa model, at the first stage, text data are tokenized and transformed to an embedding vector. For instance, a text input is tokenized and these tokens are embedded into the corresponded representation. At the second part of the RoBERTa model, these embeddings are fed to the encoder part to return an enhanced representation for each token regarding the other tokens. For example, for a text T with S tokens, a text representation for the text data T is obtained as S={sⱼ|j=1, . . . , s, sⱼ∈R^{d}}, where each eⱼ represents the information of token eⱼ regarding to the other tokens in the input text data. In some implementations, each sⱼ has the same representation dimensionality as vᵢ, in the image representation.

As another example, a BioBERT model may be used to extract feature representations for the text modality. The BioBERT model is a pre-trained language representation model for the biomedical domain. As a non-limiting example, a BioBERT model may be initialized with weights from a BERT model and then pre-trained on text data specifically associated with a biomedical domain.

As yet another example, for a text encoder, the network weights can be initialized from a BioMed-RoBERTa-base, which is a model trained on 2.68 million scientific papers from a semantic scholar corpus. This model is a language model based on RoBERTa-base architecture, which includes 12,768 hidden dimensions with 110M learnable parameters. Like the image encoder, in some instances only the last two layers of the text encoder may be trained and the rest of the network layers may be frozen.

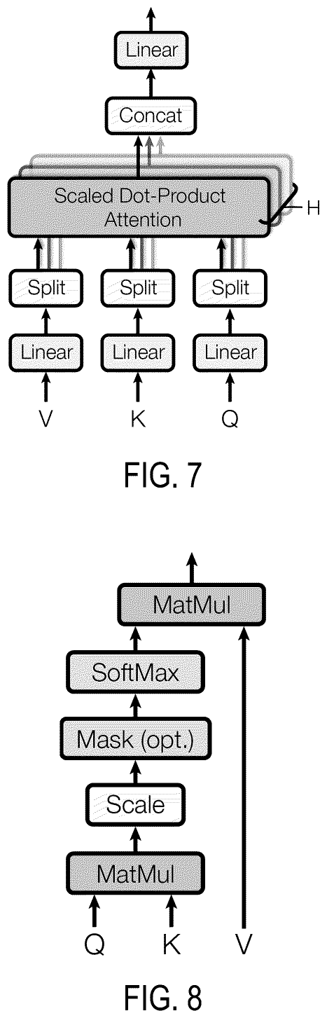

After the representation for each modality instance has been extracted, a multi-head self-attention module is applied to obtain m enhanced feature maps for extracted features from the previous stage. These m representations highlight the most significant features of each modality in relation to itself. An example multi-head self-attention module is shown in FIG. 7.

A cross-attention mechanism is utilized to attend to distinct parts of one modality given the context of another modality. An example cross-attention module is shown in FIG. 8. The application of cross-attention in the proposed approach is to attend to image regions regarding the text input tokens and vice versa. The gated memory block seeks to refine the extracted features from each modality considering the cross-attention feature maps. The input modalities are denoted as X={xᵢ|i∈[1, m], xᵢ∈ [symbol] } and Y={γᵢ|i∈[1,m],γᵢ∈ [symbol] } which X and Y can be either image or text features, where m is the number of attention heads in the multi-head self-attention module in the previous step. The cross-attention module helps to have the highlighted information for modality X related to modality Y. To achieve this goal, a suitable measurement is needed to quantify the similarity between each feature map and feature maps in another modality.

In the suggested solution, the cosine similarity is applied. The similarity between each instance in modality X and Y is determined as Eqn. (1), where sᵢⱼ denotes the similarity between the i-th feature map for modality X and the j-th from modality Y. Furthermore, it is beneficial to threshold the similarity at 0 and normalize it.

$${s}_{i j} = \frac{{x}_{i}^{T} {y}_{j}}{‖ {x}_{i} ‖ \times ‖ {y}_{j} ‖} , ∀ i ∈ [ 1 , m ] , ∀ j ∈ [ 1 , m ] {\overline{s}}_{i j} = \frac{max ( 0 , {s}_{i j} )}{{\sqrt{{Σ}_{i = 1}^{m} max ( {s}_{i j}} )}^{2}} . \qquad ( 1 )$$

To attend on set Y with respect to a given feature xᵢ in X, a weighted combination of γⱼ is defined. The definition of attention function in Eqn. 1 is a variant of the dot product attention:

$${a}_{i}^{x} = {Σ}_{j = 1}^{m} {α}_{i j} {y}_{j} , {α}_{i j} = \frac{exp ( λ {\overline{s}}_{i j} )}{{Σ}_{j = 1}^{m} exp ( λ {\overline{s}}_{i j} )} . \qquad ( 2 )$$

In Eqn. (2), the parameter λ is the inverse temperature of the softmax function. A more smooth attention function can be achieved by adjusting λ. A^{x} is defined as {αᵢ^{x}∈[1, m], αᵢ^{x} ∈ [symbol] } where each element of A^{x} captures significant parts of each xᵢ given the whole Y set as context. To refine the extracted features of X regarding the important parts of each xᵢ given Y, a memory unit can be used. The memory unit dynamically updates and refines the feature maps of X by looking to both A^{x} and X as Eqn. (3), where f(·) can be defined differently. In a non-limiting example, a gated mechanism can be adopted for f(·) as follows:

x*=f(xᵢ,αᵢ^{x}). (3)

As stated above, gated memory can assist the model in refining the feature representation regarding the shared information between two modalities. Having the gated memory in an iterative scheme can be beneficial as each iteration step, with the help of A^{X} feature representation of X can be re-calibrated. The iterative scheme can be summarized as Eqn. (4), where k is the iteration step that will be performed to refine the alignment for the next iteration:

Xₖ*=Memory(Xₖ₋₁,A^{x}). (4)

At iteration step k, the similarity score between image I and text T is calculated as follows:

$${S}_{k} ( I , T ) = α ( \frac{1}{m} {\sum }_{i = 1}^{m} {S}_{k}^{( v , v \to T )} ( {v}_{i} , T ) + \frac{1}{m} {\sum }_{i = 1}^{m} {S}_{k}^{( w , w \to I )} ( I , {w}_{i} ) ) + ( 1 - α ) ( \frac{1}{m} {\sum }_{i = 1}^{m} {S}_{k}^{( v , T )} ( {v}_{i} , T ) + \frac{1}{m} {\sum }_{i = 1}^{m} {S}_{k}^{( w , I )} ( I , {w}_{i} ) ) \qquad ( 5 )$$

where α is a learnable scalar weight parameter that balances the influence of the similarity score terms. S^{(v,v→T)} (vᵢ, T) and S^{(w,w→I)}(I, wᵢ) are defined as similarity score between image regions and text T and text tokens and image I respectively. These similarity scores can be derived as:

Sₖ^{(v,v→T)}(vᵢ,T)=sim(vᵢ,Aₖ^{v}), Sₖ^{(w,w→I)}=sim(Aₖ^{t},wᵢ) (6)

where sim( . . . ) is a suitable similarity score, such as a cosine similarity. The similarity score can be boosted by including directly the similarity between image and text as

S^{(v,T)}(vᵢ,T)=sim(vᵢ,T) and S^{(w,I)}(I,wᵢ)=sim(Aₖ^{t},wᵢ).

This can assist the model in preserving the semantic meaning of each instance while it attempts to bring paired instances closer together. To put all k steps together, the similarity score between image I and text T can be derived as follows, where k is the number of matching steps, which can be set as a hyper-parameter:

$$S ( I , T ) = {\sum }_{k = I}^{K} {S}_{k} ( I , T ) . \qquad ( 7 )$$

Training of the dual attention network model can implement a loss function that guides the training of the entire model in an end-to-end paradigm, which helps the network to have better performance and more flexibility. Accordingly, an example loss function may include two parts: the first part to draw paired image-text data closer within the shared space while making the unpaired data far from each other, and a second part to force the vision model to generate a more robust feature representation for the given images using an SLL method.

When training the dual attention network described in the present disclosure, a cross-modality retrieval loss can be computed. In a non-limiting example, an N-pairs bi-directional triplet-loss can be implemented as the following loss function:

[symbol]

=[Δ-S(Iᵢ,Tᵢ)+S(Iᵢ,Tⱼ)]₊+[Δ-S(Iᵢ,Tᵢ)+S(Iⱼ,Tᵢ)]₊. (8)

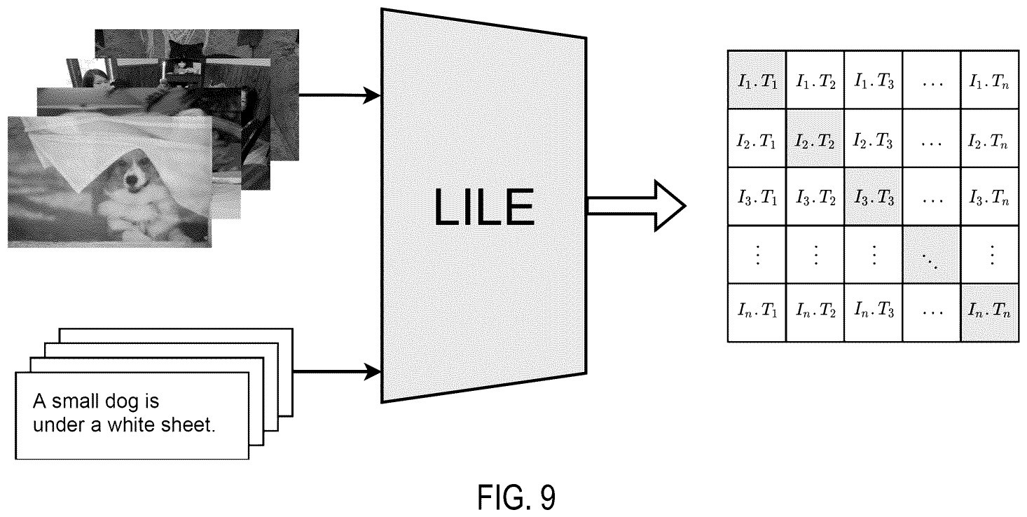

In Eqn. (8), Δ is a margin value and [x]₊=max(0, x). The term S(I,T) is defined in Eqn. (7) and measures the similarity between image I and text T. This similarity score forms a similarity score matrix S of size n×n, where S is symmetric. An example similarity matrix is shown in FIG. 9. In the training phase, n is the size of mini-batch. Images and text with a same subscript are paired instances which means the diagonal of the matrix should have the largest value compared to the other indices. This regime can be trained in an end-to-end manner.

The proposed cross-modality approach can simultaneously capture the most salient features of each modality in relation to itself and other modalities. A multi-head self-attention module was used to aid the network in gaining a better understanding of each modality. Meanwhile, to assist the model in identifying all potential alignments between two modalities, the multi-head self-attention module output is fed into a cross-attention module. This approach can aid the model in adjusting retrieved features from one modality considering the effect of the other one. Additionally, the suggested novel loss objective can assist the model in determining the optimal weight to balance both implicit and explicit sources of information used to match paired instances in different modalities.

In the training procedure, when using an H-DINO transformer as described above, the loss can be minimized with respect to θ_{S}:

$${min}_{{θ}_{s}} {\sum }_{x ∈ \{ {x}_{1}^{M} , {x}_{2}^{M} \}} {\sum }_{{{x}^{′} ∈ V}_{{x}^{′} \neq x}} - {P}_{t} ( x ) log ( {P}_{s} ( {x}^{′} ) ) . \qquad ( 9 )$$

The proposed patching scheme for the H-DINO model generates multiple views of a given image, which are assembled into a set V. This set is characterized by the inclusion of two distinct subsets of image views, differentiated by the type of augmentation applied. One subset, τ_{M}, includes views generated through the application of “Mild Augmentation”, while the other subset, τ_{H}, uses “Hyper Augmentation.” In the context of Eqn. (9), x is a selection made from the subset of views created through the τ_{M} technique. In contrast, x′ is chosen from the collective set of all views, excluding the specific view selected for x. Eqn. (9) seeks to minimize the distribution distance between these various views of the same image, which enables achieving a more robust image understanding. This minimization process assists the model in distinguishing subtle differences within the image and understanding how different elements of the image relate to each other.

The proposed loss function here can be applied on any number of patches; however, in some implementations only two patches may be used for the teacher network. When an initial teacher model, denoted by g_{θ}_{ₜ}, is not available, one may be iteratively constructed from previous versions of a student network. An approach that remarkably aligns with this framework involves applying an exponential moving averages (EMA) to the student weights and a strategy often referred to as a momentum encoder. The update rule for this strategy is described by:

θₜ←λθₜ+(1−λ)θ_{S} (10)

where λ can in some implementations undergo a gradual transition from a first value (e.g., 0.996) to a second value (e.g., 1) during the training process, following a cosine schedule. This dynamic tuning of λ orchestrates the continuous updating of the teacher network parameters, θₜ, based on the concurrently evolving student network parameters, θₛ. This symbiotic process effectively transfers learned representations and knowledge between the teacher and student networks, promoting consistent learning progress and model robustness.

After calculating the loss functions for the self-supervised task based on Eqn. (9) and the cross-modal task based on Eqn. (8), the total loss can be computed as follows:

$${ℒ}_{t o t a l} = {βℒ}_{s s l} + γ {L}_{c r oss - modal} , \qquad ( 11 )$$

$${ℒ}_{s s l} = {\sum }_{x ∈ \{ {x}_{1}^{M} , {x}_{2}^{M} \}} {\sum }_{{{x}^{′} ∈ V}_{{x}^{′} \neq x}} - {P}_{t} ( x ) log ( {P}_{s} ( {x}^{′} ) ) ,$$

$${ℒ}_{c r oss - modal} = {[ Δ - S ( {I}_{i} , {T}_{i} ) + S ( {I}_{i} , {T}_{j} ) ]}_{+} + {[ Δ - S ( {I}_{i} , {T}_{i} ) + S ( {I}_{j} , {T}_{j} ) ]}_{+}$$

The weight parameters, denoted by scalars β and γ, guide determining the impact of each loss term. These weight parameters regulate and moderate the influence of these terms on the overall loss function.

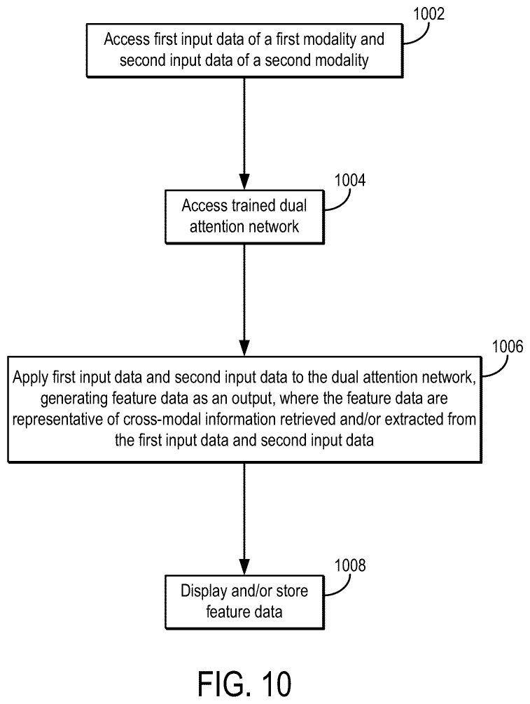

Referring now to FIG. 10, a flowchart is illustrated as setting forth the steps of an example method for generating feature data using a suitably trained dual attention network. As described, the dual attention network takes first input data associated with a first modality (e.g., text feature representation data associated with a text modality or domain) and second input data associated with a second modality (e.g., image feature representation data associated with an image modality or domain) as input data and generates feature data as output data. As an example, the feature data can be indicative of cross-modal information extracted from the first and second input data. As one example, the feature data may include similarity data estimated between the first and second input data in addition to similarity data estimated between the second and first input data.

The method includes accessing first and second input data with a computer system, as indicated at step 1002. Accessing the first and second input data may include retrieving such data from a memory or other suitable data storage device or medium. Additionally or alternatively, accessing the first and second input data may include generating such data (e.g., by inputting datasets associated with the first and second modalities to respective transformer models trained to extract feature representations for those modalities). As noted above, in some examples the first input data may correspond to a text modality and the second input data may correspond to an image modality. The text modality may be associated with a knowledge domain, such as a medical knowledge domain, a histopathology knowledge domain, or the like. The image modality may be associated with histopathology images (e.g., whole slide images, patches extracted from whole slide images), medical images (e.g., MR images, CT images, ultrasound images, PET images, etc.), or the like.

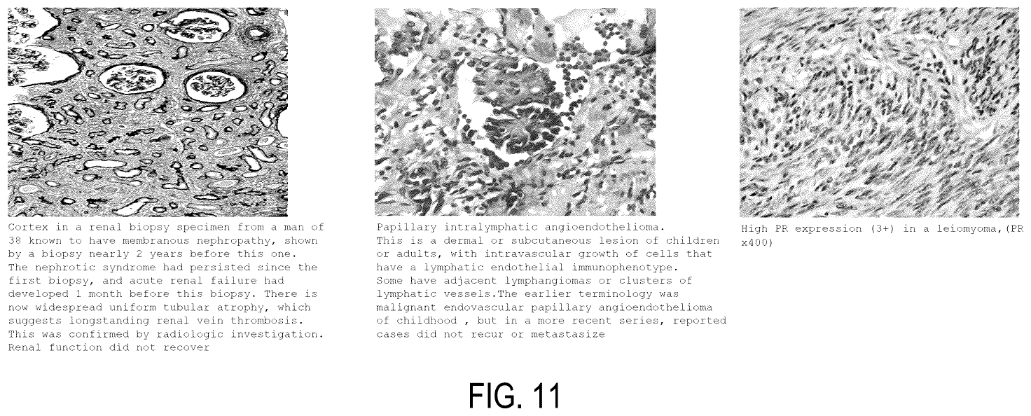

As a non-limiting example, the first input data may be text data and the second input data may be image data. An example representation of such first and second input data is shown in FIG. 11. In this example, the first input data includes morphological descriptions and diagnoses for a wide variety of tissue types and staining. The dataset images (i.e., the second input data) include histopathology images (e.g., whole slide images) corresponding to the morphological descriptions.

In some instances, when the descriptions are mostly non-uniform (e.g., ranging from 2 tokens to 484 tokens), each description may be split into sentences. Then, concatenation of the first sentence with every other sentence can be considered as new individual descriptions. By doing this, each image can have one or more than one description which can help to augment the data and uniform the descriptions.

A trained dual attention network is then accessed with the computer system, as indicated at step 1004. In general, the dual attention network is trained, or has been trained, on training data in order to extract cross-modal information and generate feature data representative of that cross-modal information.

Accessing the trained dual attention network may include accessing network parameters (e.g., weights, biases, or both) that have been optimized or otherwise estimated by training the dual attention network on training data. In some instances, retrieving the dual attention network can also include retrieving, constructing, or otherwise accessing the particular network architecture to be implemented. For instance, data pertaining to the layers in the dual attention network architecture (e.g., number of layers, type of layers, ordering of layers, connections between layers, hyperparameters for layers) may be retrieved, selected, constructed, or otherwise accessed.

As described above, the dual attention network can be constructed to include multiple modules, such as self-attention modules, cross-attention modules, and/or gated memory modules. Each of these modules may be implemented as one or more artificial neural networks. An artificial neural network generally includes an input layer, one or more hidden layers (or nodes), and an output layer. Typically, the input layer includes as many nodes as inputs provided to the artificial neural network. The number (and the type) of inputs provided to the artificial neural network may vary based on the particular task for the artificial neural network.

The input layer connects to one or more hidden layers. The number of hidden layers varies and may depend on the particular task for the artificial neural network. Additionally, each hidden layer may have a different number of nodes and may be connected to the next layer differently. For example, each node of the input layer may be connected to each node of the first hidden layer. The connection between each node of the input layer and each node of the first hidden layer may be assigned a weight parameter. Additionally, each node of the neural network may also be assigned a bias value. In some configurations, each node of the first hidden layer may not be connected to each node of the second hidden layer. That is, there may be some nodes of the first hidden layer that are not connected to all of the nodes of the second hidden layer. The connections between the nodes of the first hidden layers and the second hidden layers are each assigned different weight parameters. Each node of the hidden layer is generally associated with an activation function. The activation function defines how the hidden layer is to process the input received from the input layer or from a previous input or hidden layer. These activation functions may vary and be based on the type of task associated with the artificial neural network and also on the specific type of hidden layer implemented.

Each hidden layer may perform a different function. For example, some hidden layers can be convolutional hidden layers which can, in some instances, reduce the dimensionality of the inputs. Other hidden layers can perform statistical functions such as max pooling, which may reduce a group of inputs to the maximum value; an averaging layer; batch normalization; and other such functions. In some of the hidden layers each node is connected to each node of the next hidden layer, which may be referred to then as dense layers. Some neural networks including more than, for example, three hidden layers may be considered deep neural networks.

The last hidden layer in the artificial neural network is connected to the output layer. Similar to the input layer, the output layer typically has the same number of nodes as the possible outputs.

The first and second input data are then input to the trained dual attention network, generating output as feature data, as indicated at step 1006. For example, the feature data may include first similarity data that estimates similarity between the first input data and the second input data, and may also include second similarity data that estimates similarity between the second input data and the first input data. In the example of the first and second input data being text and image data, the first similarity data may include a text-to-image similarity matrix or map, and the second similarity data may include an image-to-text similarity matrix or map.

The feature data generated by inputting the first and second input data to the trained dual attention network can then be displayed to a user, stored for later use or further processing, or both, as indicated at step 1008. As described above, in the example where the first input data and second input data include text and image data, the feature data may be processed to generate short diagnostic reports based on the input text and images. As another example, the feature data may be processed to retrieve images based on a symptom description.

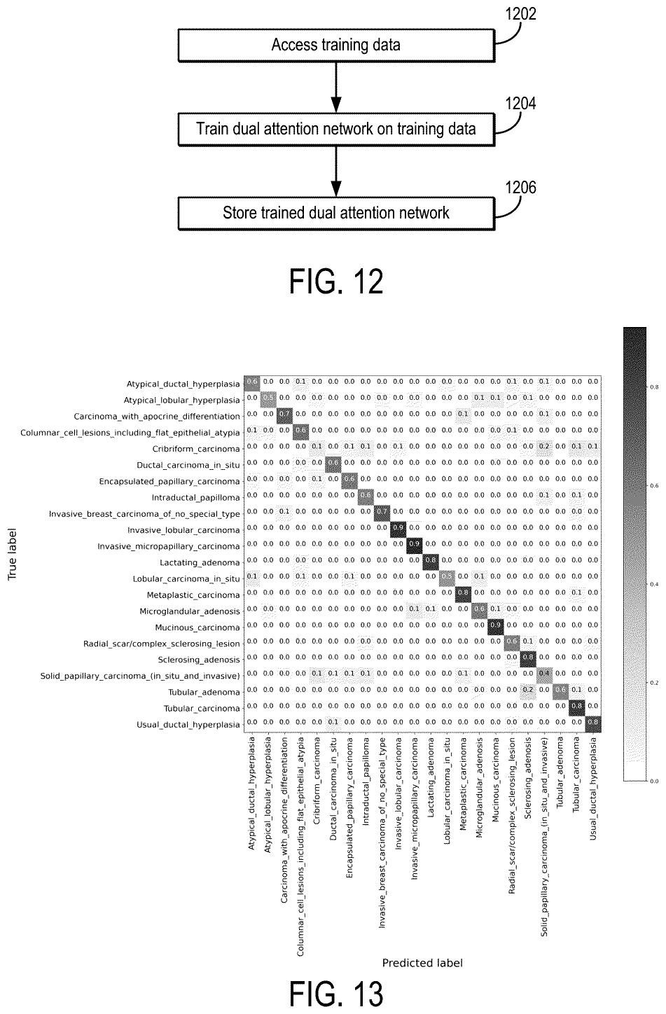

Referring now to FIG. 12, a flowchart is illustrated as setting forth the steps of an example method for training a dual attention network on training data, such that the dual attention network is trained to receive first and second input data as input data in order to generate feature data as output data, where the feature data are indicative of cross-modal information extracted from the dual modal inputs.

In general, the dual attention network can implement the architecture described above (e.g., with respect to FIG. 1). Alternatively, the dual attention network could be adapted to implement other network architectures capable of extracting cross-modal information from dual modality inputs. The dual attention network can be trained using a supervised learning approach, or other suitable learning approach such as unsupervised learning, self-supervised learning, ensemble learning, and so on.

The method includes accessing training data with a computer system, as indicated at step 1202. Accessing the training data may include retrieving such data from a memory or other suitable data storage device or medium. In general, the training data can include data representative of the first and second modalities, for which the dual attention network is being trained to extract cross-modal information. In some embodiments, the training data may include first and/or second modality data that have been labeled (e.g., labeled as containing patterns, features, or characteristics; and the like).

The method can include assembling training data from first and second modality data using a computer system. This step may include assembling the first and second modality into an appropriate data structure on which the neural network or other machine learning algorithm can be trained. Assembling the training data may include assembling first and second modality data and other relevant data. For instance, assembling the training data may include generating labeled data and including the labeled data in the training data. Labeled data may include first and/or second modality data, or other relevant data, that have been labeled as belonging to, or otherwise being associated with, one or more different classifications or categories.

The dual attention network may then be trained on the training data, as indicated at step 1204. In general, the dual attention network can be trained by optimizing network parameters (e.g., weights, biases, or both) based on minimizing a loss function. As one non-limiting example, the loss function may be the loss function in Eqn. (8). As another example, the loss function may be the loss function in Eqn. (11).

Training a neural network may include initializing the dual attention network, such as by computing, estimating, or otherwise selecting initial network parameters (e.g., weights, biases, or both). In general, during training, an artificial neural network receives the inputs for a training example and generates an output using the bias for each node, and the connections between each node and the corresponding weights. For instance, training data can be input to the initialized neural network, generating output data. The output data can be passed to a loss function to compute an error. The current network can then be updated based on the calculated error (e.g., using backpropagation methods based on the calculated error). For instance, the current network can be updated by updating the network parameters (e.g., weights, biases, or both) in order to minimize the loss according to the loss function. The training continues until a training condition is met. The training condition may correspond to, for example, a predetermined number of training examples being used, a minimum accuracy threshold being reached during training and validation, a predetermined number of validation iterations being completed, and the like. When the training condition has been met (e.g., by determining whether an error threshold or other stopping criterion has been satisfied), the current dual attention network and its associated network parameters represent the trained dual attention network. Different types of training processes can be used to adjust the bias values and the weights of the node connections based on the training examples. The training processes may include, for example, gradient descent, Newton's method, conjugate gradient, quasi-Newton, Levenberg-Marquardt, among others.

The trained dual attention network is then stored for later use, as indicated at step 1206. Storing the dual attention network may include storing network parameters (e.g., weights, biases, or both), which have been computed or otherwise estimated by training the dual attention network on the training data. Storing the trained dual attention network may also include storing the particular network architecture to be implemented. For instance, data pertaining to the layers in the dual attention network architecture (e.g., number of layers, type of layers, ordering of layers, connections between layers, hyperparameters for layers) may be stored.

In an example study, the dual attention network described in the present disclosure was collaboratively aimed at addressing cross-modality retrieval challenges in the pathology domain. The dual attention network architecture shown in FIG. 1 was utilized as the backbone of the network used in this example study. A transformer architecture was employed for both image and text encoding backbones, with self-attention modules being leveraged to highlight key aspects of images and text. Additionally, cross-attention modules were employed to extract important segments from images and text, considering their relevance to the other modality, namely text and images, respectively. Moreover, a gated memory was applied to refine extracted features for images and text regarding the output of the cross-attention module in an iterative scheme.

In this example study, a comprehensive evaluation of the disclosed systems and methods was made in comparison to alternative approaches by applying two strategies tailored to the specific nature of the datasets. The datasets in question, namely GRH and LC25000, encompass images that have been classified according to primary diagnosis and can serve as a definitive classification criterion. This can help to compare the disclosed dual attention network approach with other models like KimiaNet. As a result, in the conducted experiments on these datasets, the primary diagnosis was utilized as a descriptor for the proposed method, and to facilitate a fair comparison with other techniques without compromising the task's generality, the primary diagnosis was mapped into labels compatible with other classification approaches.

Moreover, the disclosed dual attention network approach was demonstrated to function as a bi-directional retriever for both GRH and LC25000 datasets by feeding the primary diagnosis as a description into the model. This allowed an evaluation of its performance in different settings, offering a more robust and comprehensive assessment of its efficiency and applicability.

In relation to the PatchGastricADC22 dataset, which is characterized by paired image-description entries at the WSI level, the provided descriptions were employed as text inputs. This was informed by the unique structure and specific attributes of this dataset, which is amenable to a deeper level of analysis courtesy of the substantial data encapsulated within the descriptions. Consequently, other existing cross-modal retrieval methodologies were used as points of reference for comparison and evaluation. This approach ensured that the evaluation benchmark remained constant, facilitating a rigorous comparison rooted in the specific characteristics of the dataset.

The versatility and adaptability of the disclosed dual attention network approach to different dataset configurations were highlighted, further validating its utility in various machine-learning scenarios.

A thorough delineation of the procedural specifics associated with the methodology's implementation is provided, in addition to an in-depth presentation of the experimental results derived from each of the datasets. The aim of these discussions is to illuminate the complexities of the disclosed dual attention network approach, underscore its practical utility, and confirm its resilience when applied to diverse datasets and configurations.

To conduct experiments on the GRH dataset, rigorous pre-processing steps were employed, where patches of size 448 by 448 pixels were extracted from WSIs based on FIG. 5. The student model was trained using six stringent data augmentation techniques (τ_{H}), whereas the teacher model had a relatively relaxed regiment with two less intensive augmentations (τ_{M}) from the same patch as described above. Regardless of the specific augmentation technique implemented, the final processed image was adjusted to fit a uniform size of 224 by 224 pixels. Padding was added when necessary to ensure a consistent dimension across all images. The center crop of each path was selected as the input to the model. A similar augmentation set as τ_{M} was applied on this input for uniformity. The suite of augmentations applied to this dataset encompasses a range of transformations. These include Random Crop, Random Vertical and Horizontal Flip, as well as Color Jitter, which further encompasses changes in Brightness, Contrast, Saturation, and Hue. The intensity of these augmentations is strategically regulated, being categorized under either Hyper or Mild augmentation sets based on the severity of the transformation required.

For the image representation extraction and SSL component, the student and teacher models utilized a Vision Transformer (ViT) architecture with 12 Transformer encoding blocks and 768 for the hidden states. Weight initialization for both student and teacher networks was facilitated through a pre-trained ViT model using the dataset featured in A. Riasatian, et al., “Fine-tuning and training of DenseNet for histopathology image representation using TCGA diagnostic slides,” Med. Image Analysis, 2021; 70:102032. The last six blocks of the student and teacher models are set to be trainable and frozen the rest.

The text encoder component included the BioMed-RoBERTa-based network, with fine-tuning restricted to the last two blocks. An empirical approach led to the determination of optimal values for β and γ in Eqn. (11), where the most desirable performance was observed at β=0.3 and γ=1. This configuration subtly de-emphasized the SSL component within the loss function. Given that the primary objective of the function is to identify and match paired images and texts, this adjustment allowed the model to focus more on its primary task while still benefiting from the auxiliary guidance of the SSL component.

To mitigate any potential bias and maintain the fairness of the comparison, six experimental iterations were carried out, each with a different test dataset, and assigned the rest of the dataset as train and validation sets. To further validate these comparisons, it was ensured that each test set included all 22 primary diagnoses. Model training was accomplished using the AdamW optimizer, with an initial learning rate of 1×10⁻⁶. The learning rate was adapted to decay when the evaluation metric (R@sum) ceased to improve. The configuration for the rest of the model remains consistent with the optimal parameters for the number of iteration steps (denoted as K) and the weight factor (represented by α) within the loss function. The selection of these parameters was driven to ensure that the model is effectively tuned for peak performance. This configuration continuity allows for a more robust evaluation and comparison across datasets. A comprehensive account of the findings from this setup can be found in Table 1.

TABLE 1
A comparison among the proposed method and other state-of-the-art approaches, examining
their performance in both patch-based and WSI-based retrieval tasks on the GRH dataset.

| | Text Retrieval | | | | Image Retrieval | | | | |
|---|---|---|---|---|---|---|---|---|---|
| Method | R@1 | R@3 | R@5 | R@10 | R@1 | R@3 | R@5 | R@10 | R@sum |

Patch based

| | | | | | | | | | |
|---|---|---|---|---|---|---|---|---|---|
| KimiaNet | 24.1 ± 3.6 | 43.3 ± 2.3 | 51.2 ± 2.2 | 72.3 ± 1.9 | N/A | N/A | | N/A | NA |
| BioMedCLIP | 12.6 ± 1.4 | 31.1 ± 3.7 | 42.7 ± 3.7 | 66.7 ± 5.3 | 18.4 ± 1.7 | 34.0 ± 3.2 | 41.9 ± 3.6 | 60.2 ± 4.2 | 307.6 ± 10.3 |
| LILE | 29.3 ± 4.3 | 50.0 ± 3.5 | 63.6 ± 2.0 | 80.2 ± 1.9 | 39.2 ± 4.7 | 53.6 ± 4.2 | 57.5 ± 4.6 | 61.5 ± 3.1 | 434.9 ± 15.1 |
| LILE + DINO | 31.6 ± 4.2 | 53.8 ± 3.1 | 65.4 ± 2.5 | 81.9 ± 1.0 | 40.1 ± 6.7 | 57.6 ± 5.6 | 63.9 ± 4.8 | 64.8 ± 3.1 | 459.1 ± 23.7 |
| LILE + H-DINO | 31.9 ± 3.7 | 54.2 ± 3.1 | 65.8 ± 2.4 | 82.3 ± 1.0 | 43.2 ± 6.8 | 59.8 ± 4.1 | 64.4 ± 4.8 | 65.9 ± 3.5 | 467.5 ± 18.8 |

WSI based

| | | | | | | | | | |
|---|---|---|---|---|---|---|---|---|---|
| KimiaNet | 42.3 ± 5.3 | 66.3 ± 3.2 | 70.4 ± 3.0 | 78.1 ± 2.1 | N/A | N/A | N/A | N/A | N/A |
| BioMedCLIP | 32.4 ± 2.1 | 48.4 ± 3.2 | 68.3 ± 3.4 | 74.1 ± 3.0 | N/A | N/A | N/A | N/A | N/A |
| LILE | 50.0 ± 6.1 | 70.1 ± 4.2 | 76.4 ± 2.4 | 81.8 ± 2.9 | N/A | N/A | N/A | N/A | N/A |
| LILE + DINO | 53.7 ± 7.3 | 75.7 ± 4.3 | 83.2 ± 3.1 | 90.9 ± 2.6 | N/A | N/A | N/A | N/A | N/A |
| LILE + H-DINO | 54.5 ± 6.4 | 77.3 ± 4.5 | 84.1 ± 2.3 | 92.5 ± 3.3 | N/A | N/A | N/A | N/A | N/A |

The experiments on the GRH dataset were conducted in two different schemes. As shown in Table 1, the disclosed dual attention network approach is compared with other methods in patch-based and WSI-based configurations. For the patch-based strategy, each patch needs to predict the correct primary diagnosis related to itself. In an alternate setup, the problem was approached through a WSI retrieval lens, replacing the individual patch-based retrieval. The strategy being used places considerable emphasis on majority voting for the prediction of the WSI primary diagnosis. This approach, which is largely derived from strategies employed in patch-based experiments, is centered around the accumulation of predictions made for individual patches.

The process unfolds as follows: for each patch, a determination is initially made regarding whether it has accurately retrieved its correct primary diagnosis, with various thresholds applied in the recall metric. If the true primary diagnosis is found among the top “K” retrieved diagnoses, the patch is identified as correctly retrieving the actual text. If not, the patch is assigned the label corresponding to the first retrieved primary diagnosis. Subsequently, a label is assigned to a WSI, reflecting the most common label among its constituent patches. This strategy provided a more holistic interpretation of the WSI, as it accounted for the collective intelligence of all patches in a WSI, rather than treating each patch in isolation. This perspective shift aimed at enhancing the overall retrieval performance by considering the consensus of predictions within a WSI, thus adding another dimension of robustness to the proposed method.

In the analysis of KimiaNet, a pre-trained model that had been subjected to comprehensive training using the vast TCGA dataset was employed. This model underwent further fine-tuning using the training data, with the parameters of the last fully connected layer set to be trainable. For the experiments conducted on BioMedCLIP32, their pre-trained model without any additional fine-tuning is utilized. Their model was trained on 15 million pairs of images and text extracted from articles published on PubMed.

The disclosed dual attention network approach, incorporating structure into its backbone and employing the H-DINO strategy for self-supervision, demonstrated superior performance in both patch-based and WSI-based tasks compared to other approaches. A comparative analysis, pitching the proposed LILE+DINO method against the without any self-supervision, demonstrated the potency of SSL in boosting model performance. The inclusion of SSL significantly elevated the R@sum metric, which measures the model's overall effectiveness, by a notable 32.6 margin. Furthermore, a comparison drawn between the LILE+H-DINO and the LILE+DINO methods underscored the value of implementing a patching method specifically devised for WSIs. This tailored approach to patching further enhanced the R@sum performance metric by an additional 8.4, highlighting its crucial role in optimizing the model's overall performance. This specialized approach notably improved the model's performance by providing a more robust embedding representation for the patches.

When considering text-to-WSI retrieval, the nature of the training and testing data, which are primarily patches, made it unfeasible to retrieve a WSI based on its primary diagnosis. This is due to the inherent fact that all patches extracted from the same WSI share an identical primary diagnosis, leading to a uniform result. Consequently, no results have been reported in this direction, and this aspect is noted as “N/A” (Not Applicable). Further, it is worth mentioning that the KimiaNet and other models exclusively trained for images lack the capability to retrieve images when given text, leading to an absence of reported results for these models. However, within the scope of patch-based retrieval, the models that are built upon display the compelling feature of bidirectional functionality. This characteristic highlights the distinct superiority of cross-modal retrieval models over traditional classification networks, such as KimiaNet, as they possess the capacity to operate fluidly in both retrieval directions.

It should be noted that WSIs are predominantly utilized for diagnostic purposes by medical practitioners, including oncologists and pathologists. Consequently, greater emphasis is placed on the performance of the model in the WSI-based approach as compared to patch-based results. As seen in the table, the results for the WSI-based approach considerably outperformed those for the patch-based method. This phenomenon could be attributed to the power of majority voting. A WSI was assigned to a label if the majority of its patches voted for that specific label, implying that even if some patches were incorrectly predicted, the overall true label could still be accurately identified.

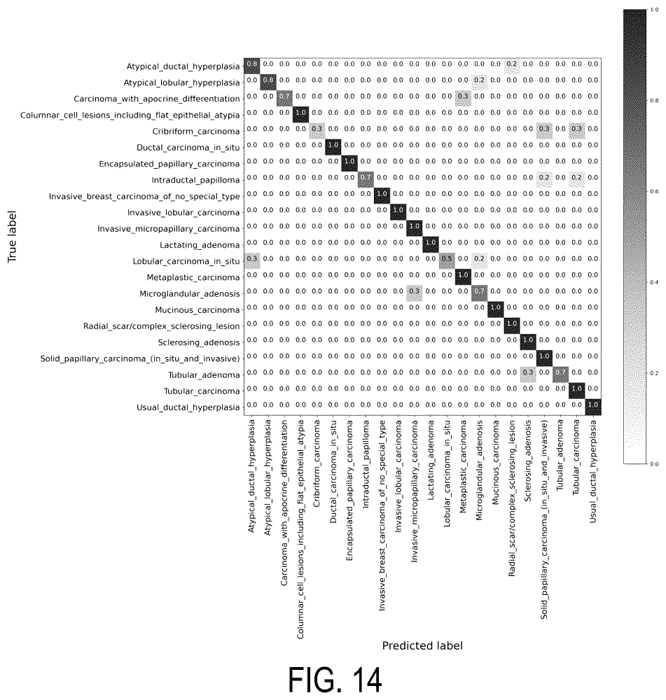

To further corroborate the efficacy of the disclosed dual attention network approach, confusion matrices for R@1, R@3, and R@5 in patch-based strategy are presented in FIG. 13. Moreover, the comprehensive performance of the model in the context of WSI retrieval at various recall thresholds—R@1, R@3, and R@5—is visualized in confusion matrices, as depicted in FIG. 14. To generate the confusion matrices that are displayed, the same methodology described in relation to Table 1 is employed. This approach allows for a consistent and comparative representation of the data across different visualizations.

These figures enable an in-depth understanding of the capability of the disclosed dual attention network approach to assign diagnoses at different levels of recall accurately. From the confusion matrices can be understood that the disclosed dual attention network approach can distinguish more of its classes, and by going from R@1 to R@3 and R@5, i.e., looking at more retrievals, one can achieve more reliable predictions. Classifying 22 primary diagnoses of breast cancer is extremely challenging as they are all related to one anatomical organ, and they exhibit many similarities in their texture. As a result, R@3 and R@5 can provide more accurate results and be practical for pathologists and doctors to be relied on. Furthermore, it is noteworthy that each primary diagnosis can occasionally be misconstrued as a different primary diagnosis. This phenomenon could potentially serve as an additional source of information for pathologists. By allowing for the possibility of alternative diagnoses, insights that might be obscured if only the correct primary diagnosis were provided can instead be unveiled, thus supporting a more comprehensive understanding of the pathology in question.

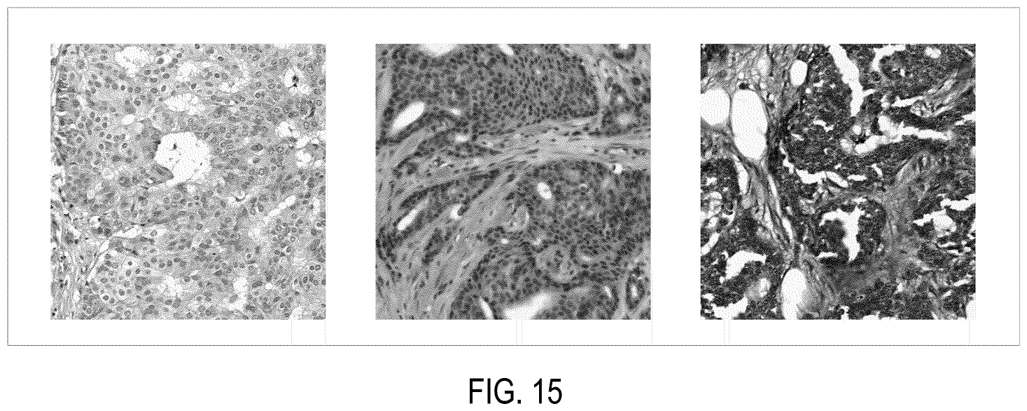

More examination of the confusion matrices reveals that two primary diagnoses, namely, Caribiform Carcinoma and Lobular Carcinoma in Situ, pose significant challenges. A contributing factor to the misclassification of Caribiform Carcinoma pertains to the limited representation of this diagnosis in the GRH dataset. With only three WSIs corresponding to this diagnosis, the dataset configuration for each training run is such that one WSI is set aside for testing, and another for validation, leaving just a single WSI for training. As depicted in FIG. 15, which showcases three random patches extracted from these three WSIs, the structural and feature variation across each WSI is quite significant. Consequently, training the model using a single WSI fails to provide a comprehensive representation of the primary diagnosis, thereby making it challenging to correctly identify this diagnosis in other WSIs.

If more data corresponding to this specific primary diagnosis were to be included in the training set, it would be reasonable to anticipate improved diagnostic accuracy. By enhancing the diversity and volume of the training data, the model would be equipped with a more holistic understanding of the diagnosis, thus improving its ability to identify and classify new instances accurately.

In another experiment, the robustness and adaptability of the disclosed dual attention network approach were demonstrated through an analysis of the generated embeddings and the real-world application of these findings. The dataset used for this experiment was sourced from the WHO (World Health Organization), which was not part of the original training set but provided real medical reports all related to the same primary diagnosis.

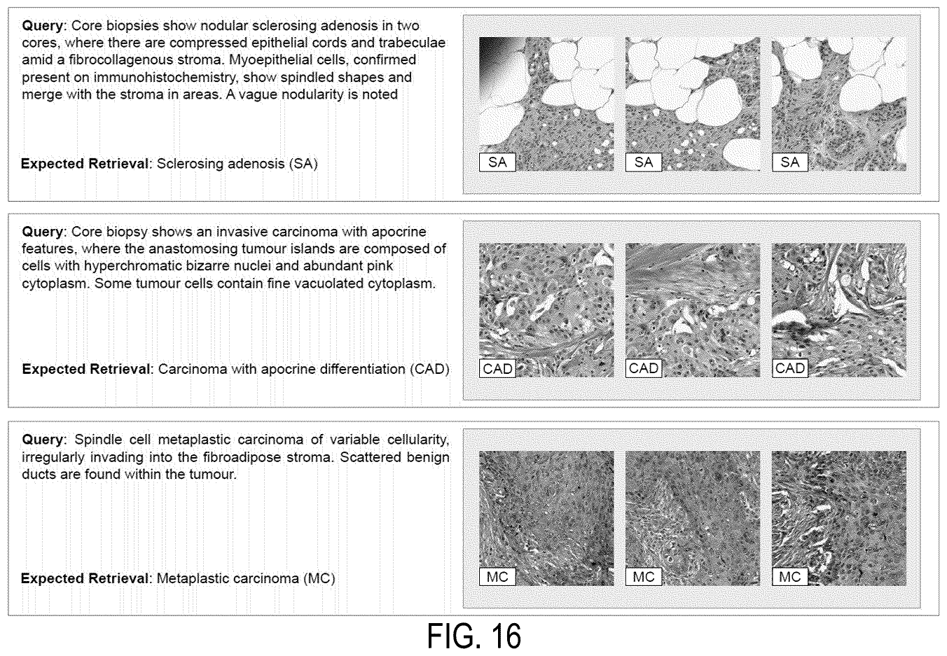

The top three results from the text-to-image (t2i) retrieval process are displayed in FIG. 16. The capability of the model to retrieve accurate and relevant images given a description of a specific primary diagnosis is clearly illustrated by these results.

These examples demonstrate how the model can identify and understand similar semantic structures between images and their corresponding descriptions. Not only does this enhance the overall precision of the model, but it also demonstrates its ability to adapt to and comprehend new, intricate concepts.

In additional sets of experiments conducted on the GRH dataset, a focus was placed on evaluating the model based on metrics more aligned with the diagnostic considerations of pathologists in order to ensure real-world clinical applications were closely reflected.

One of the key metrics, recall@K (R@k), which was reported previously, was utilized for this evaluation. The successful retrieval was defined by the premise that the correct item was found within the top K retrieved items. This measure of success was then used to cast a ‘vote’ among the patches of a WSI.

Taking the approach a step further, another strategy was adopted. This method involved the top “V” retrieved primary diagnoses for each image patch being gathered. Here, “V” functions as a voting threshold, representing the number of retrieved items deemed informative enough to contribute to the majority vote in the ultimate step. Subsequently, these items were multiplied by their corresponding normalized similarity scores. The primary diagnoses retrieved for each WSI were then organized based on the frequency of their occurrence among the top “V” diagnoses, which were weighted by their scores across all patches within that specific WSI. Following this, the recall@K evaluation was applied to ascertain if the accurate primary diagnosis was among the top “K” retrieved items. This layered evaluation technique, termed Recall@K@V, provided a multidimensional and more nuanced analysis of retrieval success and presented a more comprehensive picture of retrieval performance. Such a metric can be of assistance to pathologists when ordering immunohistochemistry; Recall@K@V is a good quantification to select the right biomarkers.

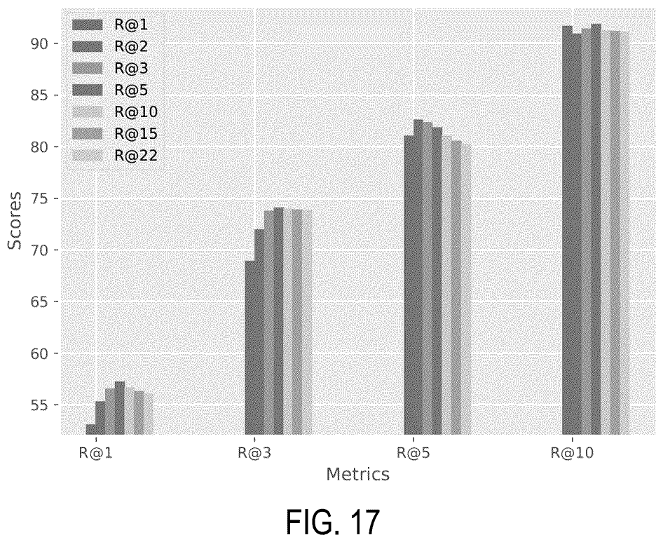

This innovative and advanced evaluation strategy facilitated the procurement of a more detailed and nuanced understanding of the disclosed dual attention network approach's performance. The results of this experiment, which offer an extensive analysis of Recall@K@V evaluations, are depicted in FIG. 17. The strategic expansion of the traditional approach allowed for a thorough and informative dissection of the model's performance.

FIG. 17 illustrates the impact of modulating the voting threshold, termed “V”, on the task of determining a WSI's primary diagnosis over a range of Recall@K values. The voting threshold specifies the number of top-retrieved items per patch taken into account in the decision-making process. By adjusting “V”, the breadth of information contributing to the final diagnostic decision can be fine-tuned, offering a nuanced perspective on the retrieval performance. The visual representation shows an initial trend of improved performance with an increasing voting threshold, underlining the value of a more inclusive decision-making process. Multiple “V” thresholds were examined to investigate this relationship, ranging from a conservative limit of 1 (i.e., only the top-retrieved item per patch contributes to the final decision) to a comprehensive limit of 22, encompassing all primary diagnoses in the database. The effects of these different thresholds are color-coded for ease of comparison.

In the processing of the PatchGastricADC22 dataset, patches of 300 by 300 pixels in size were utilized. The description of each extracted patch was linked to the corresponding WSI it was obtained from. The primary distinction in this approach is manifested in the input selection for the model, which acts as an anchor for the training of the retrieval part. Instead of a center crop being traditionally employed, variability is introduced: for each iteration in the training process, a random crop of dimensions 224 by 224 pixels is chosen from the original 300 by 300 patch and inputted into the model. This methodology is based on the assumption that a subset of the original patch, specifically the 224 by 224 pixels crop, can adequately represent the accompanying descriptive text. Moreover, this method infuses a layer of randomness into the training process, potentially fostering an enhanced generalization ability for the model. However, during the test phase, a center crop is utilized. The overall model architecture was maintained in alignment with the configuration adopted for the GRH experiments. Pre-trained models from a classification task using the dataset featured in the KimiaNet paper noted above were applied to both the student and teacher networks, while the BioMed-RoBERTa was employed as the text encoder. The only variable in this arrangement was the number of trainable blocks in the vision component, where the last eight blocks were selected for adjustment. For the text encoder, the last two blocks were set as trainable. A learning rate of 5×10⁻⁵ was chosen, and the batch size was set at 64.

Random cropping with a size of 224 by 224 for extracted different views is applied. The data augmentation methodology implemented with the GRH dataset was mirrored here, with six Hyper augmentations (τ_{H}) being applied to the student model and two less stringent ones (τ_{M}) to the teacher model. These augmentations include Random Crop, Random Vertical and Horizontal Flip, and Color Jitter (affecting Brightness, Contrast, Saturation, and Hue). The strength of these transformations was controlled, falling into either Hyper or Mild augmentation sets, depending on the desired extent of change. Optimal results were achieved when the parameters of the loss function were finely tuned, setting γ to 1 and β to 0.5. Given that the central goal of the model is to effectively locate and pair corresponding images and texts, this particular adjustment aids the model in concentrating on its main task.

Table 2 documents the performance of the model in patch-based and WSI-based retrieval compared to other methods. This table provides insights into the efficacy of the suggested method and its relative performance in comparison with other techniques.

TABLE 2
A comparison of the proposed method and other state-of-the-art
approaches on the PatchGastricADC22 dataset, evaluating their
performance in patch-based and WSI-based retrieval tasks.

| | Text Retrieval | | | | Image Retrieval | | | | |
|---|---|---|---|---|---|---|---|---|---|
| Method | R@1 | R@3 | R@5 | R@10 | R@1 | R@3 | R@5 | R@10 | R@sum |

Patch based

| | | | | | | | | | |
|---|---|---|---|---|---|---|---|---|---|
| BioMedCLIP (zero-shot) | 6.0 | 17.8 | 28.7 | 53.2 | 10.1 | 22.4 | 29.8 | 51.2 | 219.2 |
| LILE | 18.2 | 37.8 | 49.1 | 78.2 | 28.5 | 46.9 | 48.3 | 68.2 | 375.2 |
| LILE + DINO | 19.6 | 40.4 | 53.8 | 80.0 | 34.2 | 50.6 | 55.0 | 70.0 | 403.5 |
| LILE + H-DINO | 20.8 | 42.2 | 54.8 | 81.3 | 40.0 | 55.0 | 55.0 | 75.0 | 424.1 |

WSI based

| | | | | | | | | | |
|---|---|---|---|---|---|---|---|---|---|
| BioMedCLIP (zero-shot) | 14.6 | 26.2 | 32.3 | 64.5 | N/A | N/A | N/A | N/A | N/A |
| LILE | 32.4 | 46.1 | 64.3 | 85.8 | N/A | N/A | N/A | N/A | N/A |
| LILE + DINO | 35.0 | 49.5 | 66.2 | 88.3 | N/A | N/A | N/A | N/A | N/A |
| LILE + H-DINO | 36.7 | 52.3 | 67.8 | 93.2 | N/A | N/A | N/A | N/A | N/A |

To evaluate the performance of the disclosed dual attention network approach and compare it with other methods, LILE is been used as the baseline and compared LILE+H-DINO with LILE+DINO and BioMedCLIP32 which is one of the foundation models for vision and vision language tasks that trained on large paired medical data. For the sake of comparison, only the BioMedCLIP model was utilized in this instance due to its public availability and its standing as one of the SOTA techniques for cross-modal retrieval tasks in the medical field.

The same voting scheme proposed for the GRH dataset is applied for this dataset with a voting threshold of five for WSI-based retrieval tasks. The results show the efficacy of the combination of H-DINO in both patch-based and WSI-based tasks which outperformed all the other approaches. In this experiment, the pre-trained BioMedCLIP model, which was trained on a large volume of figure-caption pairs extracted from biomedical research articles in PubMed Central, was employed without further fine-tuning. To facilitate the application of the BioMedCLIP model for WSI-based retrieval, the identical procedure used for other methods was diligently implemented. The results of BioMedCLIP, as expected, are the lowest among other approaches as it is only trained with images and text extracted from research articles, and it can show the importance of data in training models. The comparative evaluation of the proposed method, LILE+H-DINO, with LILE and LILE+DINO, offers some notable insights.

Among the methods charted in the table, a distinct performance advantage was exhibited by the proposed +H-DINO. It outpaced both LILE and LILE+DINO at various recall thresholds, including R@sum, registering a lead of 48.9 and 20.6, respectively. This performance margin underscores the significance of incorporating self-supervised learning (SSL) and a tailored patching scheme in the proposed methodology, elements that played a role in enhancing retrieval accuracy.

On another note, a characteristic of this dataset is that each description is associated with all patches extracted from the corresponding WSJ. Consequently, retrieving a WSI from a description is not feasible, as the correlation doesn't lend itself to direct WSI retrieval. Therefore, the corresponding entries in Table 2 are marked as “N/A” (Not Applicable), indicating the inapplicability of certain metrics in this context.

The dataset under consideration contains authentic captions penned by pathologists, offering a practical application scenario for the proposed approach. Furthermore, the challenge escalates when distinguishing between descriptions and images pertaining to the same type of adenocarcinoma. The similarity in the nature of the images and text can blur the differentiation, thereby posing a considerable challenge. This is especially prominent when comparing the performance to that of the GRH dataset. The complex task of accurately matching the nuanced visual patterns in the images with the precise medical terminology in the captions can lead to reduced performance, reinforcing the intrinsic difficulty of the task. Nonetheless, successfully tackling this challenge can provide valuable insights for advancing cross-modal retrieval techniques in real-world clinical settings.

The LC25000 dataset was employed as another benchmark to evaluate the effectiveness of the proposed methodology. Patches of dimensions 768 by 768 pixels characterize this particular dataset. In line with model requirements, these patches were resized to 448 by 448 pixels. The methodology previously detailed for the GRH dataset was adhered to, ensuring consistent operations across all datasets. As mentioned for the PatchGastricADC22 dataset, instead of the center 224 by 224 pixels crop feed into the model, a random crop has been fed to the cross-modal retrieval network as the same characteristic applied for the LC25000 dataset. One limitation that arose with the LC25000 dataset was the absence of information regarding the WSI from which the patches were extracted. This resulted in the reporting of only patch-based retrieval outcomes for this dataset.

The preparation of views for both the student and teacher networks was undertaken in an identical manner to previous operations for the GRH and PatchGastricADC22 datasets, including the number of views and augmentations that have been applied. An exhaustive and systematic experimental analysis has ascertained optimal parameter settings tailored for this dataset. In the context of the image encoder, the student and teacher networks, fine-tuned on the last six blocks of a pre-trained ViT trained on the dataset featured in the KimiaNet paper noted above, showed the best results. On the other hand, the last two blocks of BioMed-RoBERTa were found to be most effective for the text encoder.

In terms of optimizing the loss function, the parameters were carefully calibrated. The parameter γ was set to 1, while the parameter β was set to 0.4, granting a slightly lesser emphasis to the SSL part in the loss function. A learning rate of 5×10⁻⁵ was employed, offering a good balance between training speed and model stability. The batch size was 64.

Comparisons were made between the performance of the proposed method and other existing methods. The insights derived from these comparisons have been collated and presented in Table 3.

TABLE 3
A comparison of the proposed method and other state-of-
the-art approaches on the LC25000 dataset, evaluating
their performance in patch-based retrieval tasks.

| | Text Retrieval | | | Image Retrieval | | | |
|---|---|---|---|---|---|---|---|
| | R@1 | R@3 | R@5 | R@1 | R@3 | R@5 | R@sum |
| Method | Patch based | | | | | | |
| MD | 98.4 | N/A | N/A | N/A | N/A | N/A | N/A |
| CNN and SVM | 94.0 | N/A | N/A | N/A | N/A | N/A | N/A |
| MRFO & EO | 99.6 | N/A | N/A | N/A | N/A | N/A | N/A |
| LILE | 99.3 | 100 | 100 | 100 | 100 | 100 | 599.3 |
| LILE + DINO | 99.7 | 100 | 100 | 100 | 100 | 100 | 599.7 |
| LILE + H-DINO | 99.8 | 100 | 100 | 100 | 100 | 100 | 599.8 |

The findings affirm the disclosed dual attention network approach's robustness and effectiveness when applied to this benchmark dataset, supporting its validity across various scenarios. Notably, the MD34, CNN and SVM35, and MRFO & EO36 methods are unable to be applied in the image retrieval direction, and they do not provide results for R@3 and R@5. The absence of reported results for these models further highlights this limitation. The outcomes of this experiment are noteworthy and closely matched, largely due to the straightforward nature of the task, which involves recognition among merely five distinct categories. In this context, the distinguishing factor among various methodologies primarily lies in the R@1 metric (the strict classification case), given that all methods achieved a perfect score of 100 for R@3 and R@5. Consequently, R@10 has not been reported, considering that with only five primary diagnoses, any recall threshold above 5 would invariably yield a score of 100.

Despite the high level of competition, the disclosed dual attention network approach, +H-DINO, manages to inch ahead, albeit by a narrow margin. It surpasses the performance of MRFO & EO, as cited in 36, by 0.2 for R@1, and outperforms LILE and LILE+DINO by 0.5 and 0.1, respectively, for R@1, which subsequently impacts the R@sum score.

This marginal yet significant superiority is particularly noteworthy given the circumstance where scores are concentrated near the ideal score of 100. The disclosed dual attention network approach's ability to offer this subtle performance boost in a context where the scope for enhancement is minimal indicates its capacity to yield exceptional results in more complex scenarios.

Furthermore, image retrieval results have only been provided for architectures. This is because other approaches have only been capable of functioning in a single direction—specifically, classifying images based on their primary diagnosis. This further highlights the versatility and capability of networks in the context of image retrieval tasks.

Efficient, versatile retrieval across diverse modalities, like images and texts, is an advantage for cross-modal retrieval tasks for real-world applications. Scarce labeled data makes these tasks more challenging. To address this, the disclosed dual attention network approach enable extracting robust embeddings for both image and text modalities. One aspect of this approach is the integration of SSL within knowledge distillation for cross-modal retrieval tasks and an end-to-end training regime. In this embodiment, significant feature embeddings are extracted from images using SSL, particularly using a DINO architecture. This approach is optimized with a tailored patching scheme called “harmonizing DINO” or H-DINO, designed for pathology WSIs. This refined methodology progressively identifies and captures essential features across modalities.

A multi-head self-attention module enhances the network's understanding of its modality, and the output is directed to a cross-attention module for aligning text and image modalities. An iterative alignment scheme using a memory network is employed in the proposed LILE+H-DINO strategy to augment features based on significant data segments highlighted by the self-attention and cross-attention modules.

A novel loss function was implemented, which incorporate SSL and cross-modal retrieval objectives with weighted significance to balance these interconnected objectives.

The LILE+H-DINO methodology is rigorously assessed across various datasets, consistently demonstrating superiority in terms of the Recall@K metric, even in histopathology contexts. The proposed method's potential to transform digital pathology's bidirectional cross-modality retrieval challenge is highlighted.

The effectiveness of SSL in cross-modal retrieval is demonstrated, and the integration of tailored patching for WSIs enhances performance. Harmonizing patching preserves contextual integrity and structural consistency, contributing to richer image interpretation. Performance differences between LILE, LILE+DINO, and LILE+H-DINO illustrate the benefits of merging SSL and the tailored patching scheme.

The diversity of datasets used underscores the proposed methodology's importance. The GRH breast cancer dataset, with 22 primary diagnoses, presents a formidable challenge, highlighting practical applicability. Testing on datasets created by pathologists and a less challenging dataset with five primary diagnoses confirms the disclosed dual attention network approach's adaptability and robustness.

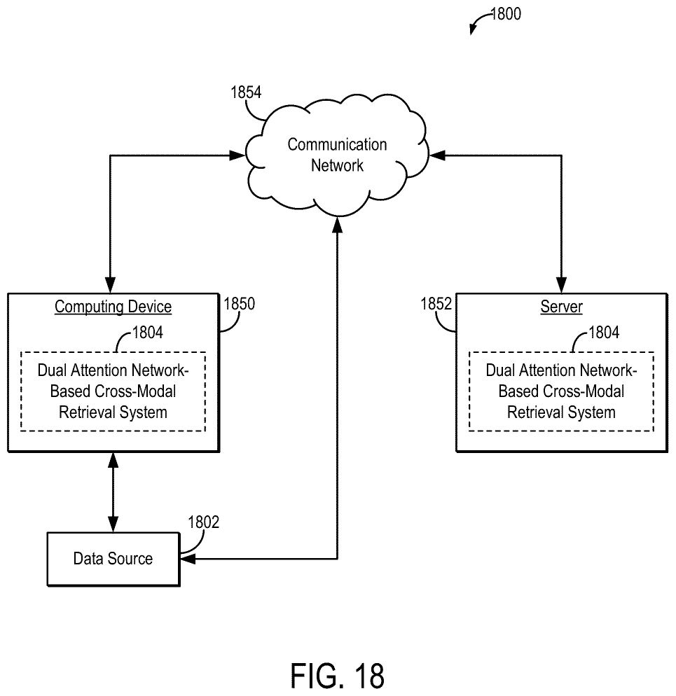

Referring now to FIG. 18, an example of a system 1800 for cross-modal retrieval from input data of different modalities (e.g., image and text) in accordance with some embodiments of the systems and methods described in the present disclosure is shown. As shown in FIG. 18, a computing device 1850 can receive one or more types of data (e.g., text data, image data, video data, voice data, molecular data, genomic data) from data source 1802. In some embodiments, computing device 1850 can execute at least a portion of a dual attention network-based cross-modal retrieval system 1804 to extract feature representations from data received from the data source 1802.

Additionally or alternatively, in some embodiments, the computing device 1850 can communicate information about data received from the data source 1802 to a server 1852 over a communication network 1854, which can execute at least a portion of the dual attention network-based cross-modal retrieval system 1804. In such embodiments, the server 1852 can return information to the computing device 1850 (and/or any other suitable computing device) indicative of an output of the dual attention network-based cross-modal retrieval system 1804.

In some embodiments, computing device 1850 and/or server 1852 can be any suitable computing device or combination of devices, such as a desktop computer, a laptop computer, a smartphone, a tablet computer, a wearable computer, a server computer, a virtual machine being executed by a physical computing device, and so on. The computing device 1850 and/or server 1852 can also reconstruct images from the data.

In some embodiments, data source 1802 can be any suitable source of data (e.g., measurement data, images reconstructed from measurement data, processed image data, text data), another computing device (e.g., a server storing measurement data, images reconstructed from measurement data, processed image data, text data), and so on. In some embodiments, data source 1802 can be local to computing device 1850. For example, data source 1802 can be incorporated with computing device 1850 (e.g., computing device 1850 can be configured as part of a device for measuring, recording, estimating, acquiring, or otherwise collecting or storing data). As another example, data source 1802 can be connected to computing device 1850 by a cable, a direct wireless link, and so on. Additionally or alternatively, in some embodiments, data source 1802 can be located locally and/or remotely from computing device 1850, and can communicate data to computing device 1850 (and/or server 1852) via a communication network (e.g., communication network 1854).

In some embodiments, communication network 1854 can be any suitable communication network or combination of communication networks. For example, communication network 1854 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 1854 can be a local area network, a wide area network, a public network (e.g., the Internet), a private or semi-private network (e.g., a corporate or university intranet), any other suitable type of network, or any suitable combination of networks. Communications links shown in FIG. 18 can each be any suitable communications link or combination of communications links, such as wired links, fiber optic links, Wi-Fi links, Bluetooth links, cellular links, and so on.

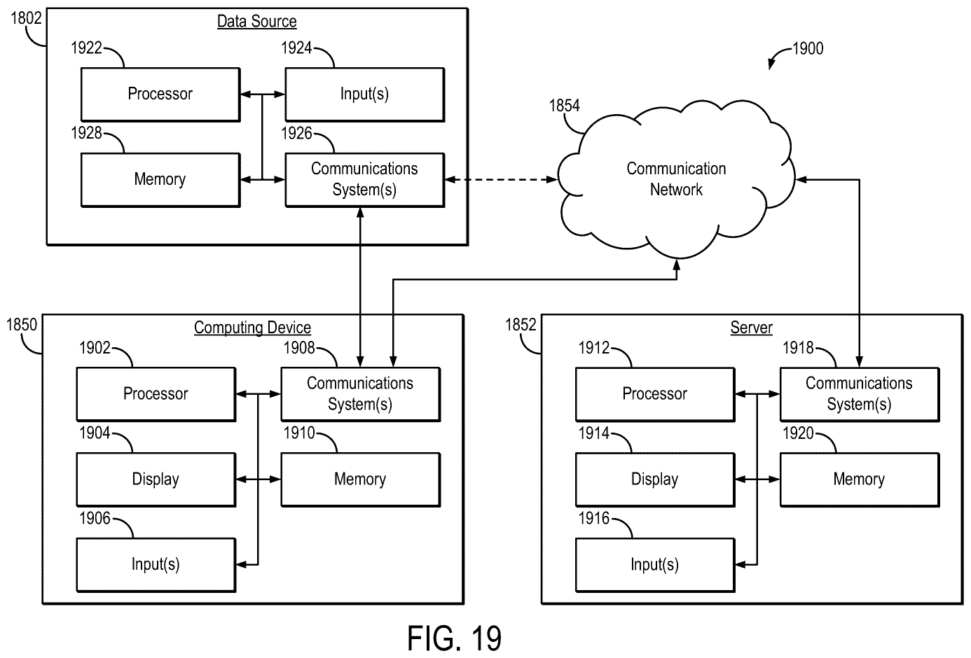

Referring now to FIG. 19, an example of hardware 1900 that can be used to implement data source 1802, computing device 1850, and server 1852 in accordance with some embodiments of the systems and methods described in the present disclosure is shown.

As shown in FIG. 19, in some embodiments, computing device 1850 can include a processor 1902, a display 1904, one or more inputs 1906, one or more communication systems 1908, and/or memory 1910. In some embodiments, processor 1902 can be any suitable hardware processor or combination of processors, such as a central processing unit (“CPU”), a graphics processing unit (“GPU”), and so on. In some embodiments, display 1904 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 1906 can include any suitable input devices and/or sensors that can be used to receive user input, such as a keyboard, a mouse, a touchscreen, a microphone, and so on.

In some embodiments, communications systems 1908 can include any suitable hardware, firmware, and/or software for communicating information over communication network 1854 and/or any other suitable communication networks. For example, communications systems 1908 can include one or more transceivers, one or more communication chips and/or chip sets, and so on. In a more particular example, communications systems 1908 can include hardware, firmware, and/or software that can be used to establish a Wi-Fi connection, a Bluetooth connection, a cellular connection, an Ethernet connection, and so on.

In some embodiments, memory 1910 can include any suitable storage device or devices that can be used to store instructions, values, data, or the like, that can be used, for example, by processor 1902 to present content using display 1904, to communicate with server 1852 via communications system(s) 1908, and so on. Memory 1910 can include any suitable volatile memory, non-volatile memory, storage, or any suitable combination thereof. For example, memory 1910 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 1910 can have encoded thereon, or otherwise stored therein, a computer program for controlling operation of computing device 1850. In such embodiments, processor 1902 can execute at least a portion of the computer program to present content (e.g., images, user interfaces, graphics, tables), receive content from server 1852, transmit information to server 1852, and so on. For example, the processor 1902 and the memory 1910 can be configured to perform the methods described herein.

In some embodiments, server 1852 can include a processor 1912, a display 1914, one or more inputs 1916, one or more communications systems 1918, and/or memory 1920. In some embodiments, processor 1912 can be any suitable hardware processor or combination of processors, such as a CPU, a GPU, and so on. In some embodiments, display 1914 can include any suitable display devices, such as an LCD screen, LED display, OLED display, electrophoretic display, a computer monitor, a touchscreen, a television, and so on. In some embodiments, inputs 1916 can include any suitable input devices and/or sensors that can be used to receive user input, such as a keyboard, a mouse, a touchscreen, a microphone, and so on.

In some embodiments, communications systems 1918 can include any suitable hardware, firmware, and/or software for communicating information over communication network 1854 and/or any other suitable communication networks. For example, communications systems 1918 can include one or more transceivers, one or more communication chips and/or chip sets, and so on. In a more particular example, communications systems 1918 can include hardware, firmware, and/or software that can be used to establish a Wi-Fi connection, a Bluetooth connection, a cellular connection, an Ethernet connection, and so on.

In some embodiments, memory 1920 can include any suitable storage device or devices that can be used to store instructions, values, data, or the like, that can be used, for example, by processor 1912 to present content using display 1914, to communicate with one or more computing devices 1850, and so on. Memory 1920 can include any suitable volatile memory, non-volatile memory, storage, or any suitable combination thereof. For example, memory 1920 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1920 can have encoded thereon a server program for controlling operation of server 1852. In such embodiments, processor 1912 can execute at least a portion of the server program to transmit information and/or content (e.g., data, images, a user interface) to one or more computing devices 1850, receive information and/or content from one or more computing devices 1850, receive instructions from one or more devices (e.g., a personal computer, a laptop computer, a tablet computer, a smartphone), and so on.

In some embodiments, the server 1852 is configured to perform the methods described in the present disclosure. For example, the processor 1912 and memory 1920 can be configured to perform the methods described herein.

In some embodiments, data source 1802 can include a processor 1922, one or more inputs 1924, one or more communications systems 1926, and/or memory 1928. In some embodiments, processor 1922 can be any suitable hardware processor or combination of processors, such as a CPU, a GPU, and so on. In some embodiments, the one or more inputs 1924 are generally configured to acquire or access text data, images, or both. Additionally or alternatively, in some embodiments, the one or more inputs 1924 can include any suitable hardware, firmware, and/or software for coupling to and/or controlling operations of the inputs 1924. In some embodiments, one or more portions of the data acquisition system(s) 1924 can be removable and/or replaceable.

Note that, although not shown, data source 1802 can include any suitable inputs and/or outputs. For example, data source 1802 can include input devices and/or sensors that can be used to receive user input, such as a keyboard, a mouse, a touchscreen, a microphone, a trackpad, a trackball, and so on. As another example, data source 1802 can include any suitable display devices, such as an LCD screen, an LED display, an OLED display, an electrophoretic display, a computer monitor, a touchscreen, a television, etc., one or more speakers, and so on.

In some embodiments, communications systems 1926 can include any suitable hardware, firmware, and/or software for communicating information to computing device 1850 (and, in some embodiments, over communication network 1854 and/or any other suitable communication networks). For example, communications systems 1926 can include one or more transceivers, one or more communication chips and/or chip sets, and so on. In a more particular example, communications systems 1926 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 1928 can include any suitable storage device or devices that can be used to store instructions, values, data, or the like, that can be used, for example, by processor 1922 to control the one or more inputs 1924, and/or receive data from the one or more inputs 1924; to generate images from data; present content (e.g., data, images, a user interface) using a display; communicate with one or more computing devices 1850; and so on. Memory 1928 can include any suitable volatile memory, non-volatile memory, storage, or any suitable combination thereof. For example, memory 1928 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1928 can have encoded thereon, or otherwise stored therein, a program for controlling operation of data source 1802. In such embodiments, processor 1922 can execute at least a portion of the program to generate images, transmit information and/or content (e.g., data, images, a user interface) to one or more computing devices 1850, receive information and/or content from one or more computing devices 1850, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for extracting features from cross-modal data from an input data source, the method comprising:
(a) accessing, with a computer system, first input data of a first modality;
(b) accessing, with the computer system, second input data of a second modality;
(c) accessing, with the computer system, a dual attention network trained on training data to extract feature data from cross-modal input data;
(d) inputting the first input data and the second input data to the dual attention network by the computer system, generating outputs as feature data comprising feature representations of the first modality and the second modality; and
(e) storing the feature data or displaying the feature data to a user with the computer system.

2. The method of claim 1, wherein the first input data comprise text data and the first modality comprises textual information, and the second input data comprise image data and the second modality comprises image information.

3. The method of claim 1, wherein the first input data comprise first feature representation data comprising feature representations extracted from a dataset of the first modality.

4. The method of claim 3, wherein the first feature representation data are extracted from the dataset of the first modality using a first transformer model.

5. The method of claim 3, wherein the second input data comprise second feature representation data comprising feature representations extracted from a dataset of the second modality.

6. The method of claim 5, wherein the second feature representation data are extracted from the dataset of the second modality using a second transformer model.

7. The method of claim 1, wherein the first modality comprises a text modality and the second modality comprises an image modality.

8. The method of claim 7, wherein the image modality comprises histopathological images.

9. The method of claim 8, wherein the dataset of the second modality comprises whole slide images.

10. The method of claim 7, wherein the second input data comprise second feature representation data comprising feature representations extracted from a dataset of the second modality using a vision transformer model.

11. The method of claim 10, wherein the vision transformer model comprises a vision transformer trained using a harmonizing distillation with no labels (H-DINO) self-supervised learning.

12. The method of claim 11, wherein the vision transformer model performs patch extraction using a larger scale than a final size of an extracted local image patch by enlarging a patch size around the local image patch to form a global image patch at the larger scale.

13. The method of claim 12, wherein the global image patch is input to a first transformer comprising a teacher model and the local image patch is input to a second transformer comprising a student model.

14. The method of claim 1, wherein the first input data comprise genomic data and the first modality comprises genomic information.

15. The method of claim 1, wherein the dual attention network comprises a first attention network associated with the first modality and a second attention network associated with the second modality.

16. The method of claim 15, wherein each of the first attention network and the second attention network include a first multi-head self-attention module that is configured to learn about the first modality and a second multi-head self-attention module that is configured to learn about the second modality.

17. The method of claim 16, wherein an output of the first multi-head self-attention module and an output of the second multi-head self-attention module are input to a cross attention module to identify alignments between the first modality and the second modality.

18. The method of claim 1, wherein the first input data comprises text data and the second input data comprises image data comprising histopathological images, further comprising generating a report based on the feature data using the computer system.

19. The method of claim 18, wherein the report comprises a diagnostic report based on cross-model features in the feature data.

20. The method of claim 1, wherein the first input data comprises text data and the second input data comprises image data comprising histopathological images, further comprising retrieving additional image data from a database based on the feature data.
